(19) United States
(12) Patent Application Publication
(10) Pub. No.: US 2010/0173991 A1
(43) Pub. Date: Jul. 8, 2010

(54) METHOD FOR THE SYNTHESIS OF A-RING AROMATIZED ACETYL MINOCYCLINES

(76) Inventors: Peter Lorenz, Magdeburg (DE); Peter Kreutzmann, Magdeburg (DE); Fritz Rothe, Magdeburg (DE); Jens Martens-Lobenhoffer, Hamburg (DE); Harry Schmidt, Lieskau (DE); Gerald Wolf, Magdeburg (DE)

Correspondence Address: JORDAN AND HAMBURG LLP, 122 EAST 42ND STREET, SUITE 4000, NEW YORK, NY, 10168, US

(21) Appl. No.: 12/669,913

(22) PCT Filed: Jun. 25, 2008
(86) PCT No.: PCT/DE2008/001055

§ 371 Date: Feb. 3, 2010

(30) Foreign Application Priority Data
Jul. 20, 2007 (DE) 10 2007 034 259.6

(51) Int. Cl. A61K 31/235; C07C 69/94; C07C 233/65; A61K 31/166; A61P 31/04

(52) U.S. Cl. 514/533; 560/139; 564/167; 514/619

(57) ABSTRACT

The aim of the invention to provide a less complex method for the production of A-ring aromatized acetyl minocyclines of the formula (I),

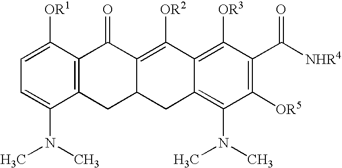

wherein R¹ to R⁵=acetyl and/or H, which can also be used on an industrial scale, is achieved in that minocycline hydrochloride is reacted with acetanhydride in the presence of a proton catcher, the reaction product is subjected to chromatographic filtration using a carrier material and an eluant, the eluant is distilled off, and the product is subsequently cleaned by recrystallization.

### BACKGROUND OF THE INVENTION

The invention relates to a method for the synthesis of A-ring aromatized acetyl minocyclines.

Minocycline (see Formula III) is a semisynthetic broad-spectrum antibiotic of the tetracycline class and in clinical practice it has been used for the treatment of, inter alia, infectious diseases of the respiratory system, the genitourinary system and the gastrointestinal tract, of different skin diseases, such as acne vulgaris, rosacea, and of trachoma, chlamydia-conjunctivitis and Lyme disease for many years.

Minocycline inhibits the protein biosynthesis by bonding to the ribosomal 30S-subunit thus avoiding the access of aminoacyl-t RNS to the RNS ribosomal complex and consequently the extension of the peptide chain.

Apart from the already known antibiotic effect of minocycline another biological effect of the substance has become the focus of research recently. Initial examinations show that minocycline obviously has a protective effect for different inflammation processes and neurodegenerative diseases (Yong, V. W., Wells, J., Giuliani, F., Casha, S., Power, C., and Metz, L. M. (2004). The promise of minocycline in neurology. Lancet Neurol 3, 744-751).

Thus, the use of minocycline is presently recommended for the clinical treatment of progressing inflammation processes, such as inflammatory rheumatoid arthritis (Furst, D. E. (1998). Update on clinical trials in the rheumatic diseases. Curr Opin Rheumatol 10, 123-128). Inflammation processes play a major role in the pathogenesis of neurodegenerative diseases, for example of the Alzheimer's disease, Parkinson's disease and multiple sclerosis as well as of post-traumatic injuries of the brain and spinal cord.

DE 38 81 024 T2 reveals a method for the production of tetracycline derivates, such as minocycline. Said method comprises several stages, uses catalysts on a carrier and organic solvents and is performed under pressure, and only the dehalogenation and hydration processes are carried out within one step.

This production method has the disadvantage that much effort and consequently high costs are required. Moreover, selenium-containing alloys are used, amongst others, as catalyst.

The patent WO 2005/070878 discloses A-ring aromatized tetracycline derivates and a method for the production thereof.

The production of A-ring aromatized tetracyclines, as described in this publication, is principally successful but requires several reaction stages and an extensive cleaning of the reaction products by preparative HPLC that is not suited for an industrial and thus efficient extraction of the active substances, e.g. on gram or kilogram scale.

Due to the biological and pharmacological effects found the use of minocycline for the treatment of neurodegenerative diseases is very interesting. The examination of minocycline as a guide structure is an attractive approach in the search for neuroprotective agents.

The minocycline guide structure can be optimized and improved in two directions:
1. Improvement of the pharmacokinetic, e.g. by realizing a prodrug concept.
2. Abolishment of the antibiotic activity, if it is not relevant for the effect as a neuroprotective substance, in order to exclude a selection pressure on microorganisms towards a resistance development.

The expert knows the prodrug concept as a method that is used for the chemical change of hydrophilic active molecules in such a way that, on the one hand, their lipohiles and thus their absorbing capacity through membranes increase and, on the other hand, the actual active molecules are only developed from a precursor (prodrug) in the cells. Here, the precursor (prodrug) acts, amongst other functions, as a carrier.

A prodrug is per definition a substance or drug that is not or almost not pharmacologically effective without metabolism and only becomes an active agent by the metabolism in the body. Prodrugs are of strategic importance in such cases in which only a small and less selective amount of the actual agent reaches the target site.

The prodrug concept targets an improvement of the pharmacokinetic properties of the active molecules, for example, to improve their resorption capacity/bioavailability or to allow the blood-brain-barriers passability for a psychopharmacon or neuroprotective drug.

Due to the free hydroxyl(OH)- or amino(NH₂)-hydrophilic groups active molecules, such as minocycline, often exhibit a good solubility in water that supports their pharmaceutical formulation but they can hardly penetrate membranes.

The introduction of apolar protecting groups is a possibility to reduce their polarity. The acetyl group (CH₃CO) has been proven as an apolar protecting group (‘prodrug moiety’). After separation it develops free acetic acid in the cells that is a natural metabolite of the cell metabolism. Both OH- and NH₂-groups can be masked by acetyl groups. The polarity of the acetylated molecules is similar to the one of biological membranes but a stronger interaction with lipids and consequently an increased diffusion into the tissue (carrier effect) are reached. After the absorption of the acetylated prodrug by the cells the acetyl groups are separated by unspecific esterases and thus the actual active molecule is endogenously released and can become active here.

Furthermore, the prodrug is a controlled-release form of the active molecule that implies a delayed release and a more favorable pharmacological behavior.

Only few neuroprotective active molecules derived from minocycline have been described in literature so far. Although they are based on the natural substance model they have not been used for following a prodrug concept up to now [Wang, R., Du, Y., and Liu, Zhou, Z., Wang, H., Wang, X., (J. (2004). Synthesis and neuroprotective activity of novel C4, C7 derivates in tetracycline series. J Chin Pharm Sci 13, 217-220].

### BRIEF DESCRIPTION OF THE INVENTION

The task of this invention is to avoid the disadvantages of the state of the art by providing a less complex method for the production of A-ring aromatized acetyl minoclyclines of the Formula I that can also be used on an industrial scale.

For this purpose, this invention recommends to perform a single or multiple acetylation of the guide structure with a simultaneous dehydration and aromatization of the A-ring in one step to obtain a neuroprotective agent.

Surprisingly, in a single-step reaction of minocycline hydrochloride with acetic acid hydride (acetanhydride) in the presence of an organic proton catcher A-ring aromatized single- or multiple-acetylated minocyclines of Formula I (wherein R¹ to R⁵=acetyl and/or H) are obtained by the acetylation of minocycline hydrochloride, the preparative chromatographic cleaning or separation of the reaction products and the subsequent further cleaning by means of the recrystallization of the corresponding products from an ethyl acetate/gasoline-kerosene mixture.

Such A-ring aromatized acetyl minocyclines of Formula I (wherein R¹ to R⁵=acetyl and/or H) release A-ring aromatized minocycline of Formula I (wherein R¹ to R⁵═H) after metabolism in the organism and said substance shows a cell- or neuro-protective effect but not an antibiotic one.

Single- or multiple-acetylated A-ring aromatized minocyclines of Formula I (wherein R¹ to R⁵=acetyl and/or H) are produced by the reaction of minocycline hydrochloride with acetanhydride in the presence of an organic proton catcher. In this reaction, an equimolar excess of acetanhydride or the organic proton catcher is used and they simultaneously act as a solvent for the minocycline hydrochloride.

It is also possible to use equimolar amounts of the starting materials according to the number of acetyl groups that are to be introduced, and reduced amounts of acetanhydride and of the proton catcher are to be replaced by an inert solvent, if required. Suitable inert solvents for the starting materials are, for example, chloroform, methylene chloride, nitromethane, acetonitrile, acetone, sulfolane, dimethylformamide or dimethylsulphoxide.

Pyridine is preferably used as the proton catcher and the reaction is advantageously performed at a temperature ranging from 4 to 100° C., preferably at 75° C. or below the boiling point of the reaction mixture. other proton catchers can also be used instead of pyridine, for example primary, secondary or tertiary amines or carboxylic acid amides.

The reaction is normally performed at normal pressure by stirring the reaction mixture. For this purpose, a glass-reaction apparatus provided with a return condenser is used. The produced solvent vapors are condensed and continuously returned into the reaction mixture.

But the inventive method can also be carried out at a reduced or increased pressure. The application of an increased pressure is particularly useful if the reaction shall be performed at a temperature at which the solvent boils at normal pressure.

Surprisingly, the two new substances pentaacetyl cyclin (A-ring aromatized pentaacetyl minocycline) and tetraacetyl cyclin (A-ring-aromatized tetraacetyl minocycline), which have not been published so far, can be obtained in one reaction step in the inventive method.

FIG. 1 shows as an example the LC(HPLC) chromatogram and the corresponding mass spectra of the two main components of a reaction mixture after the addition of excess acetanhydride. The high selectivity of the reaction for the pentaacetylated product (Formula II and FIG. 1B) can be seen. The selectivity for the relevant target product can be even further increased by optimizing the reaction conditions and then the cleaning steps described below can be simplified or are not required any longer.

In the inventive method explained here a chromatographic separation or cleaning of the product by means of VLC (vacuum liquid chromatography) is performed after the end of the reaction time.

Depending on the technical design, the VLC method can also be applied on an industrial scale. For such a reaction the mixture is preferably given on a carrier material that has been pre-conditioned by gasoline-kerosene (boiling point: 40-60° C.) or another hydrocarbon, such as n-pentan, n-hexan, cyclohexan or isobutan, and is positioned in a Buchner funnel provided with a fritted base, a chromatographgy column or a suspended vessel with screen bottom. Now, the eluent is guided through the carrier material (stationary phase) by means of vacuum sucks (VLC) or overpressure.

The elution of the target compounds is performed afterwards by applying a solvent-gradient mixture consisting of a hydrocarbon and a carboxylic is acid ester and the polarity of the mixture is increased during the elution by higher portions of the carboxylic acid ester.

In the method introduced here, a mixture of gasoline-kerosene and ethyl acetate is preferably used for the elution of the target compounds. But it is also possible to use eluent mixtures of hydrocarbons with other carbonic acid esters, such as methyl formate, n-butyl acetate or dimethyl carbonate.

Silica gel (e.g. silica gel 60), aluminum oxide, ‘reversed phase’ silica gel or Sephadex are used as the carrier material (stationary phase).

After the separation of the corresponding fractions that contain the enriched target compounds the eluent is distilled off at a reduced pressure by means of a rotation evaporator and the residual of one of the solvent mixtures mentioned above is recrystallized.

For the recrystallization in the inventive method, the corresponding target compound is dissolved first in ethyl acetate under slight heating and then a crystallization is initiated by the addition of gasoline-kerosene, i.e. by the reduction of the solubility product.

After the end of the crystallization and cooling of the mixture at 4° C. for some hours, the target compound is filtered in a filter funnel and the residual solvent is removed by drying in vacuum.

The novel substances pentaacetyl cyclin (A-ring aromatized pentaacetyl minocycline of Formula II) and tetraacetyl cyclin (A-ring aromatized tetraacetyl minocycline of Formula IV) indicated in the following as examples and produced by the inventive method have been characterized by mass spectroscopy (LC/MS, HR/MS), UV/VIS spectroscopy as well as by ¹H- and ¹³C-NMR.

So, FIG. 2 shows the UV/VIS spectra of the two just mentioned substances as an example and FIG. 3 shows the ¹H-NMR of tetraacetyl cyclin of Formula IV.

The test of the cell-protecting properties of the substances produced in the inventive method was carried out, exemplarily for a pure form of pentaacetyl cyclin of Formula II, by means of an astrocyte damaging model. This model is used for the examination of neurodegenerative diseases caused by oxidative stress or for the test of cell-protecting substances that can avoid or reduce such damage. In the exemplary model astrocytes are damaged by hydrogen peroxide (H₂O₂) and afterwards the functionality and morphology of the cells and mitochondria are microscopically assessed. An overproduction of H₂O₂ as reactive oxygen species (ROS) is considered to be, amongst others, the cause for many neurodegenerative processes and diseases in the CNS, such as stroke, Alzheimer's disease Parkinson's disease, post-traumatic injuries of the brain and spinal cord. Astonishingly, already much lower doses of pentaacetyl cyclin of Formula II could considerably reduce the damage of the mitochondria compared to the comparative substance minocycline hydrochloride (Example 3 and FIG. 4). This finding is surprising and shows the excellent activity properties of the substance that has been tested as an example.

The test of the antibiotic effect of the pentaacetyl cyclin obtained in the inventive method was performed by using an E. coli strain (Embodiment 4). Unlike minocycline hydrochloride, pentaacetyl cyclin does not show an antibiotic activity any longer. Thus, a further desired biological effect has been surprisingly achieved.

### BRIEF DESCRIPTION OF THE DRAWINGS

In the following, examples explain the invention in detail in schematic drawings.

They show:

FIG. 1: Chromatogram of an LC/MS analysis of the inventive reaction mixture before cleaning, wherein

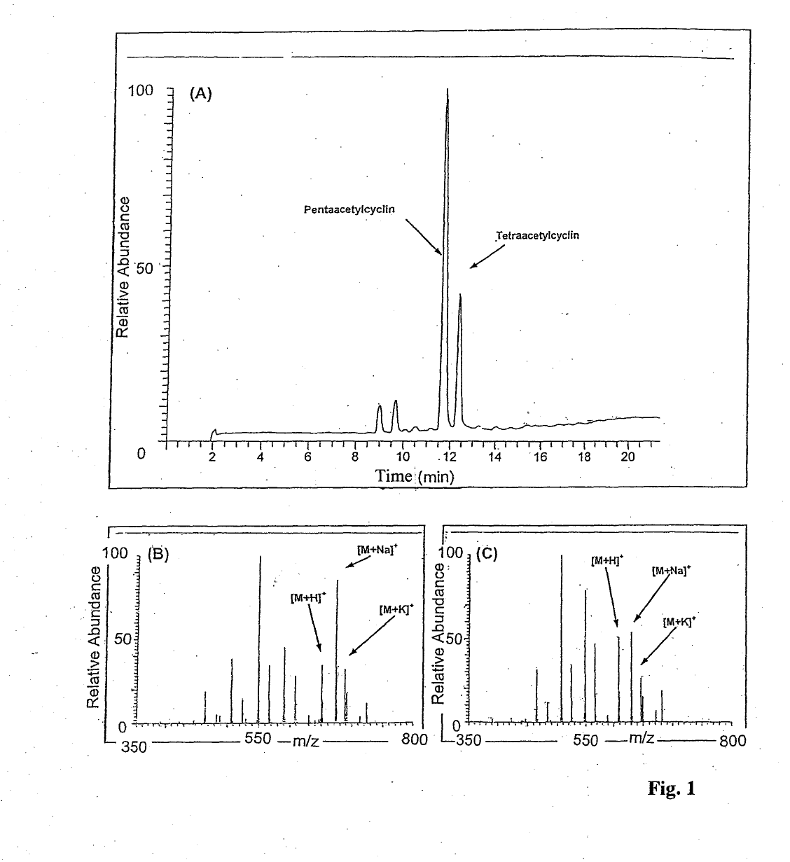

(A)—analytic LC-chromatogram of the reaction mixture,

(B)—mass spectrum of the pentaacetyl cycline (analysis of the peak at 11.69 min), and

(C)—mass spectrum of the tetraacetyl cyclin (analysis of the peak at 12.37 min),

FIG. 2: UV/VIS spectra of pentaacetyl cyclin (A) and tetraacetyl cyclin (B), produced according to the inventive method, and

FIG. 3 ¹H-NMR spectrum of tetraacetyl cyclin (in DMSO-d6) produced according to the inventive method, wherein (A) is an enlarged representation of the range from 2.0-3.6 ppm.

### EMBODIMENT 1

### Synthesis of Pentaacetyl Cyclin of Formula II

Minocycline hydrochloride (0.60 g; 1.215 mmol) was dissolved under ice-cooling (4° C.) in 12 ml pyridine (11.73 g; 148.37 mmol) and then 12 ml acetanhydrid (12.98 g, 127.18 mmol) was added under stirring. Afterwards, the mixture was stirred at 4° C. during 30 minutes, at room temperature during 2 hours and at 75° C. during 30 minutes. The reaction product was cleaned by means of vacuum liquid chromatography (VLC). For this purpose, silica gel 60 (75 g) was condensed in a glass filter funnel under vacuum extraction and preconditioned with gasoline-kerosene (boiling range: 40-60° C.). The reaction mixture was loaded on the so prepared chromatography column and eluded with a gasoline-kerosene/ethyl acetate gradient (4/1, vol/vol to 100% EtOAc). Fractions that contained the reaction product were combined. After distilling off the solvent in the rotation evaporator a residual (518.5 mg) was obtained that was recrystallized from an ethyl acetate/gasoline-kerosene mixture and finally delivered the almost pure pentaacetyl cyclin as a light-yellow solid. The crude yield was 237.5 mg. A further VCL was performed for the final cleaning of the product. The pure yield was 181.9 mg (23% of theory). The analytic test of the individual fractions was made by means of thin film chromatography (DC with fluorescence indicator, polygram SIL G/UV₂₅₄, company Macherey & Nagel) with silica gel 60 as the stationary phase and gasoline-kerosene/ethyl acetate (1/3, vol/vol) as the mobile phase. For cell culture experiments or for tests of the antibiotic effect strain solutions of the substance were produced in DMSO, sterilely filtered by means of nalgene nylon filters (pore size of 0.22 μm) and stored at −20° C. until their use.

### Analytical Data Pentaacetyl Cyclin (A-Ring Aromatized Pentaacetyl Minocycline):

R_{f} (DC)=0.50

UV-VIS (λₘₐₓ in MeOH): 330 nm (log ε 4.27)

LC/MS (positive ion mode): 723 [M+C₃H₆O₂]⁺, 688 [M+K]⁺, 672 [M+Na]⁺, 650 [M+H]⁺, 608 [650-acetyl+H]⁺, 590 [608-H₂O], 565 [608-acetyl]⁺, 548 [590-Acetyl+H]⁺, 506 [548-Acetyl+]⁺

HR-ESI-MS (negative ion mode): (M−H⁻) found 648.2198825, C₃₃H₃₄O₁₁N₃, dev. 2.5 ppm

HR-ESI-MS: (M-Acetyl) found 606.2093178 (M-acetyl), C₃₁H₃₂O₁₀N₃, dev. 1.2 ppm

HR-ESI-MS (positive ion mode): (M+Na⁺) found 672,21638, C₃₃H₃₅O₁₁N₃Na, dev. 2.4 ppm

¹H-NMR (DMSO-d6): δ (ppm) 2.16 (s, CH₃); 2.23 (s, CH₃); 2.27 (s, CH₃); 2.28 (s, 2 CH₃); 2.56 (m, CH); 2.66 (s, N(CH₃)₂); 2.73 (s, N(CH₃)₂); 2.76 (m, CH₂, superimposed by N(CH₃)₂ signal); 3.48 (m, CH₂); 7.00 (d, J=8.6 Hz, aromat. CH); 7.29 (d, J=8.2 Hz, aromat. CH); 11.08 (br s, NH)

¹³C-NMR (DMSO-d6): signals, selection δ (ppm) 13.9; 20.2; 20.6; 24.6; 31.7; 42.4; 43.9; 59.5; 122.0; 124.5; 139.9; 148.5; 161.7; 167.2; 167.8; 168.1; 168.9; 169.8

### EMBODIMENT 2

### Synthesis of A-Ring Aromatized Tetraacetyl Minocycline of Formula IV

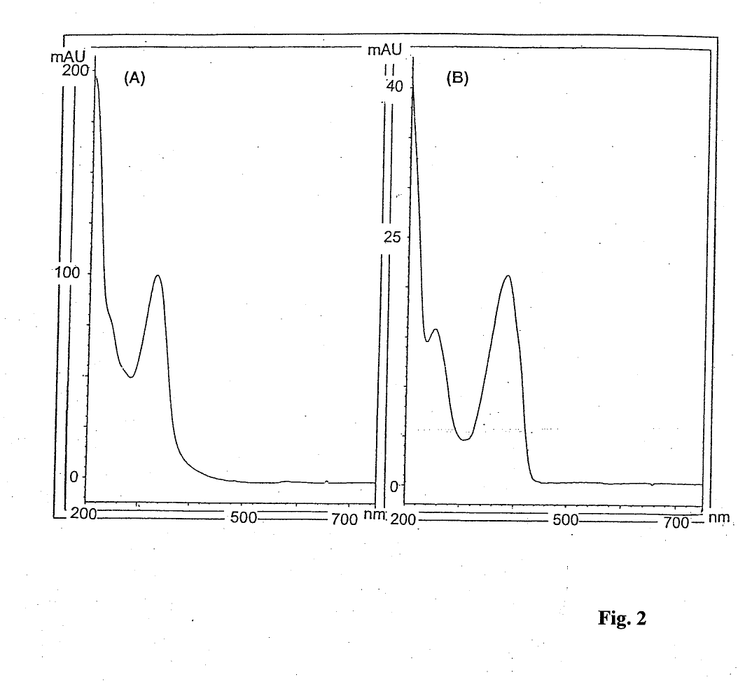

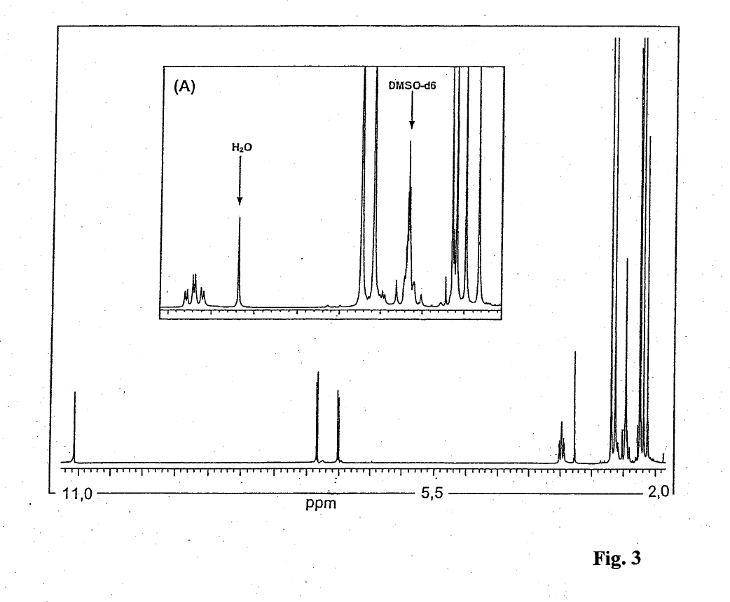

Tetraacetyl cycline was chromatographically isolated as a by-product from the reaction mixture described in the embodiment and afterwards purely obtained from the corresponding fraction after distilling off the solvent and recrystallizing from a gasoline-kerosene/ethyl acetate mixture. Due to the bathochrome shift of the UV bands compared to the one of pentaacetyl cyclin of Formula II, the enol than, according to Formula IV, is the most probable one (FIG. 2). The other (not enolic) OH- or NH₂-groups in the minocycline show a higher basicity and therefore they are preferably acetylated. The ¹H-NMR spectrum (FIG. 3) supports the structure proof.

### Analytical Data Tetraacetyl Cyclin (A-Ring Aromatized Tetraacetyl Minocycline):

R_{f} (DC)=0.57

UV-VIS (λmax): 252.381 (log ε 4.27)

LC/MS (positive ion mode): 681 [M+C₃H₆O₂]⁺, 646 [M+K]⁺, 630 [M+Na]⁺, 608 [M+H]⁺, 566 [608-acetyl+H]⁺, 548 [566-H₂O]⁺, 524 [566-acetyl+H]⁺, 506 [524-H₂O]⁺

¹H-NMR (DMSO-d6): δ (ppm) 2.16 (s, CH₃); 2.22 (s, CH₃); 2.27 (s, CH₃); 2.29 (s, CH₃); 2.49 (dt, J=13.7 Hz, CH₂); 2.63 (m, J=4.5; 13.6 Hz; CH); 2.66 (s, N(CH₃)₂)_{;} 2.72 (s, N(CH₃)₂); 3.50 (dt, J=4.5; 14.9 Hz; CH); 7.04 (d, J=8.6 Hz; aromat. CH); 7.37 (d, J=8.6 Hz; aromat. CH); 11.10 (s, NH)

### EMBODIMENT 3

### Test of the Cell or Neuroprotective Effect Properties at the Astrocytes-Mitochondria Model

Astroglia cells, i.e. non neoplastic embryonal astrocyte cell line of the rat [Chamaon, K., Kirches, E., Kanakis, D., Braeuninger, S., Dietzmann, K., and Mawrin, C. (2005). Regulation of the pituitary tumor transforming gene by insulin-like-growth factor-I and insulin differs between malignant and non-neoplastic astrocytes. Biochem Biophys Res Commun 331, 86-92] were cultivated on glass cover slips coated with poly-D-lysin at the bottom of culture dishes at 37° C. during 18 hours (5% CO₂). 2 ml DMEM (PAA Laboratories GmbH Pasching, Austria) were used as the culture medium in each reaction and the sowing density was 0.3×10⁶ cells/2 ml. First, the cell cultures were pre-incubated in different concentrations (1.0; 5.0 or 25.0 μM) of minocycline hydrochloride or pentaacetyl cyclin (Formula II) during 30 minutes. The sole addition of the solvent DMSO (2 μl/culture dish) under identical incubation conditions was used as the untreated control. Then, 1.0 or 3.0 mM H₂O₂ (final concentration) was added and a further incubation was performed for 2 hours. The correspondingly treated cells, which have only been cultivated with 1.0 or 3.0 mM H₂O₂ or only with minocycline hydrochloride or pentaacetyl cyclin in the indicated concentrations, were used for comparison. After replacing the culture medium by 2 ml on HEPES buffers (10 mM HEPES, 140 mM NaCl, 5 mM KCl, 2 mM CaCl₂, 10 mM D-glucose, pH 7.4) tempered at 37° C. the cells were further incubated during 30 minutes. Afterwards, 40 nM MitoTracker® Orange CMTMRos (1 μl of an 80 μM strain solution of the oxidized form, order no. M7510; λ_{Excitation}=554 nm, λ_{Emission}=576 nm; Molecular Probes Inc., Eugene, USA) were added. After an incubation period of 30 minutes at 37° C. the cell cultures were fixated in 4% paraformaldehyde (m/vol in HEPES buffer) during 30 minutes, were rinsed three times with 0.1 M PBS for 10 minutes to remove the fixation agent and then the glass cover slips with the fixated cells were attached on slides in ImmuMount®. A Zeiss Axiophot fluorescence microscope with CCD camera (AxioCam MRc) was used for the visual inspection and documentation.

Untreated control cells represent themselves polygonal cell bodies with 2-3 elongated processes and appear as a cellular net if the cell density is sufficient. Filiform mitochondria are distributed in the cytoplasm up to the inside of the cell processes. After incubation with 1 mM H₂O₂ (without minocycline or pentaacetyl cyclin) during a period of 2 hours most of the cells are damaged and show a clear retraction of their processes. Thus, the cell bodies lose their polygonal shape and tend to a rounded form. As a result of fusion the mitochondria are extremely reduced and dislocated towards the cell core. These cell damaging processes are intensified with 3 mM H₂O₂. The density of the adhering cells is reduced, the cell bodies are much more rounded and the even more shortened mitochondria are accumulated close to the cell core. The addition of 25 μM minocycline hydrochloride to the cells that have been damaged by H₂O₂ before has a protective effect. The cells mainly maintain their polygonal shape and the mitochondria are less shortened. Surprisingly, the use of pentaacetyl cyclin showed that the H₂O₂-induced damage of the mitochondria is considerably reduced even at the much lower concentration of 1.0 μM. This protective effect of the A-ring aromatized pentaacetyl minocycline is an improvement compared to minocycline hydrochloride that shows a similar protective activity only from an amount of 25 μM. In the test system and the concentrations used here minocycline hydrochloride and pentaacetyl cyclin do not damage the cells.

### EMBODIMENT 4

### Test of the Antibiotic Effect of Pentaacetyl Cyclin and Minocycline Hydrochloride in the Agar Diffusion Test

The liquid culture (cultivated in LB-Lennox-L-Broth-Base for 24 hours) of an E. coli suspension (150 μl) of the C 600 hfc strain was evenly distributed on LB agar (Lennox-L-Agar, Gibco) by means of a sterile Drigalski spatula. Afterwards, sterile filter paper sheets (diameter 5.3 mm) that have been soaked with sterile DMSO solutions and different concentrations (100 μM to 2.5 mM) of minocycline hydrochloride or pentaacetyl cyclin (Formula II) were placed on the breeding ground. After the incubation of the agars at 37° C. for 24 hours, the antibiotic activity of the substances was assessed on the basis of the diameters of the inhibiting areolas that become visible due to the missing opacification by bacteria. The different sizes of the inhibiting areolas show that minocycline hydrochloride, depending on its concentration, inhibits the growth of E. coli. However, pentaacetyl cyclin does not show any growth inhibition even in the highest tested concentration of 2.5 mM and consequently it does not have an antibiotic effect against the E. coli strain used.

All elements presented in the description and the subsequent claims can be essential for the invention both as single elements and in any combination.

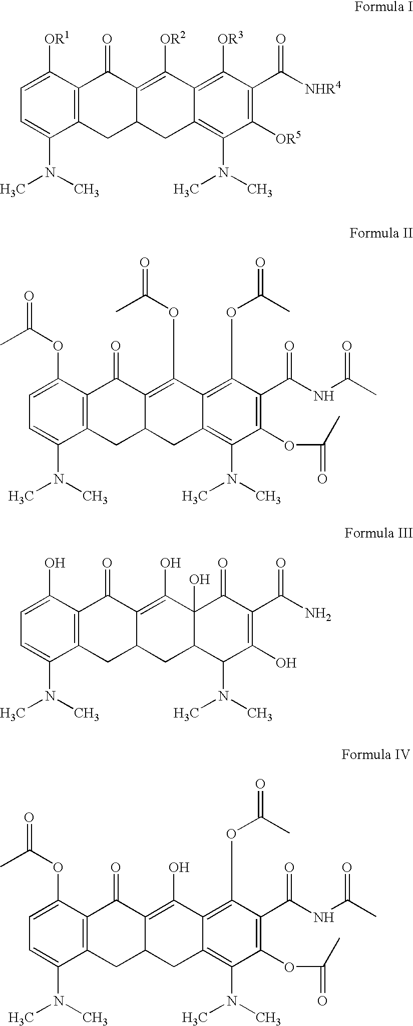

1. Method for the production of an A-ring aromatized acetyl minocycline 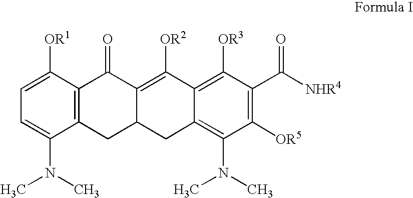
wherein R¹ to R⁵=acetyl and/or H, comprising reacting minocycline hydrochloride with acetanhydride in the presence of a proton catcher, performing a single or multiple acetylation of the guide structure under simultaneous aromatization of the A-ring, chromatographically cleaning the reaction product by using a carrier material and an eluent, distilling off the eluent and afterwards cleaning the reaction product by recrystallization wherein at least one R=acetyl.

2. Method according to claim 1, wherein an organic base is used as the proton catcher.

3. Method according to claims 1 or 2, wherein the base is a primary, secondary or tertiary amine.

4. Method according to claim 1 or 2, wherein the proton catcher comprises pyridine.

5. Method according to claim 1 or 2, wherein the acetanhydride and the proton catcher are used in excess amounts or equimolar according to the number of the acetyl groups to be introduced.

6. Method according to claim 1 or 2, wherein the reaction is performed in an inert solvent.

7. Method according to claim 6, wherein the solvent is chloroform, methylene chloride, nitromethane, acetonitrile, acetone, sulfolane, dimethylformamide or dimethylsulphoxide.

8. Method according to claim 1 or 2, wherein the reaction is performed at a temperature ranging from 4 to 100° C., at normal pressure or overpressure.

9. Method according to claim 1 or 2, wherein the A-ring aromatized acetyl minocycline comprises A-ring aromatized pentaacetyl minocycline of Formula II 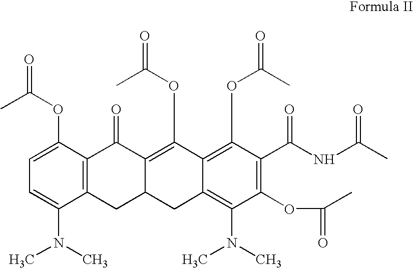
as a main product.

10. Method according to claim 1 or 2, wherein the acetyl groups in the molecule number 5 or less.

11. Method according to claim 1 or 2, wherein A-ring aromatized tetraacetyl minocycline of Formula IV 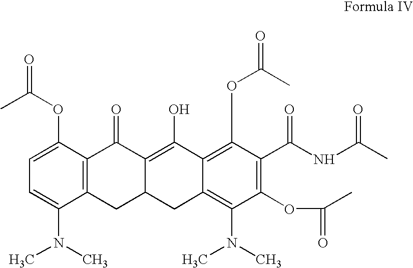
comprises a by-product.

12. Method according to claim 1 or 2, wherein the chromatographic cleaning of the reaction products is performed with silica gel 60, aluminum oxide, ‘reversed-phase’ silica gel or Sephadex as a carrier material.

13. Method according to claim 1 or 2, wherein the chromatographic cleaning is performed in a packed bed, in a chromatographic column, in a Buchner funnel provided with a fritted base or in a suspended vessel with screen bottom insert.

14. Method according to claim 1 or 2, wherein the eluent comprises a mixture of ethyl acetate and gasoline-kerosene and the recrystallization of the reaction product is carried out in a mixture of ethyl acetate and gasoline-kerosene.

15. Method according to claim 1 or 2, wherein the eluent comprises methyl formate, n-butyl acetate, dimethyl carbonate, n-pentane, n-hexane, cyclohexane or isobutane and the recrystallization of the reaction product is carried out in methyl formate, n-butyl acetate, dimethyl carbonate, n-pentane, n-hexane, cyclohexane or isobutane.

16. (canceled)

17. (canceled)

18. A pharmaceutical composition for treating a neurodegenerative disease caused by at least one of oxidative stress or mitochondrial damage comprising a compound produced by the method of claim 1 in an amount effective for treatment of said disease.

19. A method of treating a neurodegenerative disease caused by at least one of oxidative stress or mitochondrial damage comprising administering to a person suffering said disease a pharmaceutical composition of claim 18.
